(19) United States
(12) Patent Application Publication
(10) Pub. No.: US 2024/0207487 A1
(43) Pub. Date: Jun. 27, 2024

(54) VIABLE BIOENGINEERED ALLOGENEIC CELLULARIZED SKIN CONSTRUCTS THAT SECRETE SOLUBLE FACTORS ASSOCIATED WITH REGENERATIVE WOUND HEALING

(71) Applicant: Stratatech Corporation, Madison, WI (US)

(72) Inventors: Allen R. Comer, Madison, WI (US); Kenneth R. Gratz, Madison, WI (US); B. Lynn Allen-Hoffmann, Madison, WI (US)

(21) Appl. No.: 18/568,672

(22) PCT Filed: Jun. 8, 2022
(86) PCT No.: PCT/US2022/032726

§ 371 Date: Dec. 8, 2023

Related U.S. Application Data
(60) Provisional application No. 63/208,309, filed on Jun. 8, 2021.

(51) Int. Cl. A61L 27/38; A61L 27/24; A61L 27/54; A61L 27/60

(52) U.S. Cl. CPC A61L 27/3813; A61L 27/24; A61L 27/54; A61L 27/60; A61L 2300/254; A61L 2300/414

(57) ABSTRACT

The present disclosure is directed to viable bioengineered allogeneic cellularized construct.

### FIELD OF THE DISCLOSURE

The present disclosure is directed to viable bioengineered allogeneic cellularized skin constructs that deposit human extracellular matrix proteins and secrete soluble factors associated with regenerative wound healing.

### BACKGROUND

Patients who suffer severe thermal burns face a long and painful recovery. When surgical intervention is required, such as in some deep partial-thickness burns, a patient's own skin or skin from a cadaver may be grafted to the wounded area. Although autografts reduce the chance of rejection that comes with use of cadaver skin, it requires the patient to endure further trauma. What is needed are grafts that promote healing and have a low chance of being rejected by a patient.

### SUMMARY OF THE DISCLOSURE

Among the various aspects of the disclosure may be a viable bioengineered allogeneic cellularized construct for topical use at a wound site, the construct comprising allogenic cultured keratinocytes, dermal fibroblasts, and murine collagen. In some aspects, the construct deposits and/or actively produces human extracellular matrix (ECM) proteins and secretes soluble factors associated with regenerative wound healing.

In some aspects, the construct deposits human extracellular matrix proteins and secretes soluble factors after thawing from cryopreservation. The construct provides sustained secretion of the soluble factors one to four hours after thawing.

In some aspects, secreted soluble factors are human growth factors, cytokines, interleukins, and/or matrix metalloproteinases. The secreted soluble factors induce inflammation, proliferation, granulation, and remodeling of existing matrix to promote regenerative skin healing at the wound site. The secreted soluble factors are selected from the group consisting of bFGF, GM-CSF, HGF, IL-1α, IL-6, IL-8, IL-10, MMP-1, MMP-3, MMP-9, PIGF, SDF-1α, TGF-β1, VEGF-A, and combinations thereof. The secreted soluble factors are present in a range of pg/cm²/h at 1 to 168 hours after in vitro reculture.

In some aspects, construct organizes a stratified ECM reminiscent of intact skin and/or a stratified ECM substantially similar to intact human skin. The construct of may comprises one or more ECM proteins that are present in intact human skin, or a plurality ECM proteins that are present in intact human skin. The human ECM proteins are selected from the group consisting of ECM proteins with spatial distributions of fibrillar collagens I and III, collagen VI, decorin, laminin-332, and combinations thereof. The construct comprises a structurally organized dermal layer and construct may include type I and type III fibrillar collagens in conjunction with supporting proteins, type VI collagen, and decorin. The construct comprises a basement membrane and dermal-epidermal junction and the construct may include a spatial distribution of type IV collagen and laminin-332.

In some aspects, the secretion of the soluble factors is increased after meshing the allogenic cellularized construct.

Other aspects of the present disclosure include a method of treating an adult patient for deep partial-thickness (DPT) burns comprising applying an allogeneic cellularized construct to a DPT wound site, the allogenic cellularized construct comprising allogenic cultured keratinocytes, dermal fibroblasts, and murine collagen. The allogeneic cellularized construct may deposit human ECM proteins and secretes soluble factors associated with regenerative wound healing.

In some aspects, the allogeneic cellularized construct provides an established, organized ECM that increases, accelerates, and/or facilitates reepithelilization. The increase, acceleration, and or facilitation of reepithelization of the construct may be relative to an autograft of comparable surface area.

In an aspect, the method further includes meshing the allogenic cellularized construct prior to applying the allogeneic cellularized construct to the DPT wound site.

In another aspect, the method further includes thawing a cryopreserved allogeneic cellularized construct to applying the allogeneic cellularized construct to the DPT wound site. The allogeneic cellularized construct provides sustained secretion of the soluble factors one to four hours after thawing.

Further aspects of the present disclosure include a method of treating an adult patient for thermal burns comprising administering to an intact dermal element of the adult patient in need thereof an allogeneic cellularized construct, wherein the construct comprises allogenic cultured keratinocytes, dermal fibroblasts, and murine collagen, and wherein the allogeneic cellularized construct deposits and/or actively produces human ECM proteins and secretes soluble factors associated with regenerative wound healing.

Additional aspects of the present disclosure include a method of treating an adult patient for deep partial-thickness burns comprising administering to an intact dermal element of the adult patient in need thereof an allogeneic cellularized construct, wherein the construct comprises allogenic cultured keratinocytes, dermal fibroblasts, and murine collagen, and wherein the allogeneic cellularized construct deposits and/or actively produces human ECM proteins and secretes soluble factors associated with regenerative wound healing.

Yet further aspects of the present disclosure include a method of treating adult patients in need of surgical intervention comprising applying an allogenic cellularized construct to a surgically prepared wound bed, wherein the construct comprises allogenic cultured keratinocytes, dermal fibroblasts, and murine collagen, and wherein the allogeneic cellularized construct deposits and/or actively produces human ECM proteins and secretes soluble factors associated with regenerative wound healing.

### BRIEF DESCRIPTION OF THE FIGURES

FIG. 1 depicts manufacturing and components of a viable bioengineered allogeneic cellularized construct of the present disclosure.

FIG. 2A is a negative control image for immunostaining using neonatal foreskin with the primary antibody omitted resulted in no visible ECM protein staining in the epidermal layer (E) or dermal layer (D).

FIG. 2B shows neonatal foreskin immunostained with goat anti type-III collagen resulted in distinct staining in the dermal layer. Dashed line indicates the junction between the epidermal and dermal layers.

FIG. 2C shows positive control immunostaining of the nascent dermal epithelium of the viable bioengineered allogeneic cellularized construct using rat anti type I collagen on the viable bioengineered allogeneic cellularized construct which shows a loosely organized ECM. Scale bar=200 μm.

FIGS. 3A-3D shows indirect immunofluorescence of proteins typically present in the dermal layer and epidermal/dermal junction during the manufacture of the viable bioengineered allogeneic cellularized construct and after cryopreservation. FIG. 3A shows a representative fluorescence image for the detection of ECM proteins. Indirect immunofluorescence with commercial antibodies was used to detect ECM proteins (collagen, laminin-332, and decorin) on cross sections of StrataGraft. Nuclei were stained with DAPI. FIG. 3B shows StrataGraft synthesized fibrillar collagen types I and III, representing the predominant structural components.

FIG. 3C shows human collagens were deposited and organized by the cellular components of StrataGraft, including accumulation of type VI collagen and decorin, which function in vivo to guide assembly of collagen structure. FIG. 3D shows the spatial distribution of collagen IV and laminin-332 indicates that StrataGraft organized a basement membrane zone and dermal-epidermal junction.

FIGS. 4A-4C show an analysis of relative levels of secreted factors from the viable bioengineered allogeneic cellularized construct after thawing and after meshing. Viable bioengineered allogeneic cellularized constructs were thawed and recultured for the indicated time (horizontal axis), and secretion rate for each analyte was determined at each time point. FIG. 4A shows the results of the secretion of interleukins. FIG. 4B shows the results of the secretion of growth factors and cytokines. FIG. 4C shows the results of the secretion of matrix metalloproteinases. Error bars represent ±1 standard deviation from mean values. bFGF, basic fibroblast growth factor; GM-CSF, granulocyte-macrophage colony-stimulating factor; HGF, hepatocyte growth factor; IL, interleukin; MMP, matrix metalloproteinase; PIGF, placental growth factor; SDF-1α, stromal cell-derived factor 1α; FGF-β1, transforming growth factor β1; VEGF-A, vascular endothelial growth factor A.

FIG. 5 shows an in vitro experimental setup for measuring biomolecule factor secretion. Protein levels for an array of soluble wound-healing factors were quantified in conditioned media collected from unmeshed and meshed StrataGraft.

FIG. 6 shows characterization of the ECM.

### DETAILED DESCRIPTION

The present disclosure encompasses compositions comprising allogeneic cultured keratinocytes, methods of preparing such compositions for human application, and methods of administering such compositions. In particular, such compositions deposit human extracellular matrix proteins and secrete soluble factors associated with regenerative wound healing.

An allogeneic cultured keratinocyte composition of the present disclosure is a viable bioengineered allogeneic cellularized construct that may be used to support durable wound closure and regenerative healing of thermal burns requiring surgery. It contains a fully stratified epithelial layer of metabolically active allogeneic human keratinocytes grown on a collagen matrix embedded with normal human dermal fibroblasts.

The viable cells of a composition of the present disclosure (e.g., fibroblasts, NIKS cells, etc.) are metabolically active and secrete a spectrum of growth factors, chemotactic factors, cytokines, inflammatory mediators, enzymes, and host defense peptides that, after the composition is applied to a wound, may condition the wound bed, promote tissue regeneration and repair, and reduce infection. The viable cells of a composition of the present disclosure, namely keratinocytes and dermal fibroblasts, are karyotypically stable. In addition, the keratinocytes and dermal fibroblasts do not exhibit anchorage-independent growth.

In an exemplary embodiment, the composition is StrataGraft®™. In another exemplary embodiment, the composition is ExpressGraft™.

Wound healing involves complex and coordinated spatiotemporal activities in a variety of cells types to promote hemostasis, proliferation, migration, reepithelialization, and ECM deposition and remodeling to restore the integrity of the skin. Burn wounds present unique considerations since they typically involve tissue eschar, blistering, systemic damage, and in some cases wound progression that causes damage beyond the initial injury site. The depth of the burn wound is a critical factor in determining the extent of clinical intervention required for healing. Burns extending through the epidermis and papillary dermis, referred to as superficial partial-thickness burns, may be treated nonoperatively and generally heal without scarring. In contrast, deep partial-thickness (DPT) burns extend through the lower or reticular dermis and typically require more extensive clinical interventions, such as surgical placement of an autograft harvested from the healthy skin of the patient to achieve wound closure.

Autografting is a commonly used clinical treatment in severe DPT burn wounds. Since this approach requires the excision of healthy skin from a donor site on the patient, significant pain, morbidity, and other challenges are associated with donor site harvest; this drives the pursuit of autograft-sparing regenerative therapies. Laboratory synthesized cellular constructs, transplanted onto excised or debrided wound sites, encourage existing viable dermal and epidermal cells to repopulate the wound area with cells that eventually restore the epidermal and dermal extracellular matrix and skin barrier function.

The viable bioengineered allogeneic cellularized construct consists of a viable, differentiated, fully stratified epidermal layer of NIKS® human keratinocytes, grown on a collagen hydrogel matrix embedded with normal human dermal fibroblasts (NHDF). At the end of the manufacturing process, the viable bioengineered allogeneic cellularized construct is cryopreserved to maintain viability and metabolic activity during storage.

In some embodiments, up to 90%, 91%, 92%, 93%, 94%, or 95% of patients with DPT thermal burns who receive the viable bioengineered allogeneic cellularized construct treatment may exhibit complete wound closure (measured as 95% re-epithelialization with no drainage). In other embodiments, in patients with DPT thermal burns, up to 90%, 91%, 92%, 93%, 94%, or 95% of treatment sites may achieve durable wound closure (defined as 100% reepithelialization without drainage or dressing requirements) after 3 months. In other embodiments, the viable bioengineered allogeneic cellularized construct is well tolerated in full-thickness wounds and does not induce acute immunogenic responses at the treatment sites.

### Fully Stratified Epithelial Layer

A composition of the present disclosure comprises a fully stratified epithelial layer that is epidermis-like and/or substantially similar to epidermal tissue. The fully stratified epithelial layer comprises human keratinocytes. In some embodiments, the fully stratified epithelial layer comprises NIKS cells. NIKS® cells were deposited with the ATCC (CRL-12191) and are described in further detail in U.S. Pat. Nos. 5,989,837 and 6,964,869, the disclosures of which are incorporated herein by reference.

A fully stratified epithelial layer may encompass NIKS® cells engineered to express a variety of exogenous nucleic acids. Expressly contemplated are NIKS® cells engineered to express an exogenous gene encoding a VEGF protein (e.g., VEGF-A, etc.), an exogenous gene encoding a hypoxia-inducible factor (e.g., HIF-1A, etc.), an exogenous gene encoding an angiopoietin (e.g., ANGPT1, etc.), an exogenous gene encoding a cathelicidin peptide or a cleavage product thereof (e.g., hCAP-18, etc.), an exogenous gene encoding a beta-defensin (e.g., hBD-3, etc.), an exogenous gene encoding a keratinocyte growth factor (e.g., KGF-2, etc.), an exogenous gene encoding a tissue inhibitor of metalloproteinases (e.g., TIMP-1, etc.), an exogenous IL-12 gene, as well as exogenous nucleic acid sequences encoding other antimicrobials, growth factors, transcription factors, interleukins and extracellular matrix proteins. As non-limiting examples, see for instance, U.S. Pat. Nos. 7,498,167, 7,915,042, 7,807,148, 7,988,959, 8,808,685, 7,674,291, 8,092,531, 8,790,636, 9,526,748, 9,216,202, and 9,163,076, and US 20190030130, the disclosures of which are incorporated herein by reference. Compositions comprising NIKS cells engineered to express an exogenous nucleic acid encoding a desired protein produce a greater amount of that protein (e.g., at least 10%, at least 20%, at least 30%, etc. more) than a composition comprising NIKS cells that do not contain the exogenous nucleic acid.

In some embodiments, the fully stratified epithelial layer has a thickness of about 75 μm to about 120 μm, as measured by histology. For example, the fully stratified epithelial layer may have a thickness of about 75, 80, 85, 90, 95, 100, 105, 110, 115, or 120 μm, as measured by histology.

### Dermal Equivalent Layer

A composition of the present disclosure encompasses a dermal equivalent layer that is dermis-like and/or substantially similar to epidermal tissue. The dermal equivalent layer has a top surface and a bottom surface, and comprises human dermal fibroblasts within a matrix.

### Dermal Fibroblasts

A composition of the present disclosure encompasses a dermal equivalent layer that comprises dermal fibroblasts. In most embodiments, the dermal fibroblasts are human fibroblasts. In exemplary embodiments, the human dermal fibroblasts are primary normal human dermal fibroblasts.

### Matrix

A composition of the present disclosure encompasses a dermal equivalent layer that comprises a matrix. The matrix of the dermal equivalent layer comprises human collagen and optionally, murine type I collagen. In some embodiments, the matrix of the dermal equivalent layer comprises human extracellular matrix proteins, such as human type I collagen, human type III collagen, human type IV collagen, human type VI collagen, and optionally, murine type I collagen.

The collagen present in the dermal equivalent may include type I murine collagen. Alternatively, the only collagen present in the dermal equivalent may be produced by cells of the skin substitute (e.g., human dermal fibroblasts). The matrix may further comprise additional biomolecules produced by the cells contained therein. In an exemplary embodiment, the dermal layer is composed of normal human dermal fibroblasts embedded within a matrix produced and organized by the fibroblasts (e.g. an extracellular matrix). In some iterations of this embodiment, there is no non-human collagen in the dermal equivalent layer. In other iterations of this embodiment, there is up to about 85% non-human collagen in the dermal equivalent layer. In particular iterations, the non-human collagen is murine. In another iteration, the non-human collagen consists of murine collagen and includes no other non-human collagen material. In another exemplary embodiment, the dermal equivalent layer is composed of normal human dermal fibroblasts embedded in a gelled-collagen matrix that contains purified murine type I collagen. For the avoidance of doubt, in this embodiment, although the murine type I collagen is gelled to give the dermal layer its primary structure, the normal human dermal fibroblasts embedded therein may produce and contribute collagen (and other biomolecules) to the matrix. Accordingly, the collagen matrix may comprise both murine type 1 collagen and human collagen (produced from the human dermal fibroblasts).

A composition of the present disclosure may comprise human type I collagen and murine type I collagen, wherein the murine type I collagen is not more than 90% by weight of total collagen in the composition. For instance, in some embodiments, the murine type 1 collagen is about 60% to about 90% by weight of the total type collagen in the composition.

### Allogeneic Cellularized Scaffold/Construct

An allogeneic cultured keratinocyte composition of the present invention may be a viable bioengineered allogenic cellularized construct. As used herein, an “allogeneic cellularized construct” is a sterile composition of the present disclosure that has been cryopreserved, prepared for application to a patient, and optionally, dimensioned for placement over a wound bed of the patient. The phrase “allogeneic cellularized construct” may be used interchangeably herein with the terms “allogeneic cellularized scaffold”, “construct”, or “viable bioengineered allogeneic cellularized construct”.

An allogeneic cellularized construct comprises allogenic cultured keratinocytes, dermal fibroblasts, and murine collagen as detailed above.

### Manufacturing

Suitable manufacturing processes for producing a composition of the present disclosure have been previously described in the art. See, for instance, U.S. Pat. Nos. 7,498,167, 7,915,042, 7,807,148, 7,988,959, 8,808,685, 7,674,291, 8,092,531, 8,790,636, 9,526,748, 9,216,202, 9,163,076, 10,091,983, and US 20190030130, the disclosures of which are each incorporated by reference in their entirety.

In each of the above embodiments, mouse cells are not used in the manufacture of a composition of the present disclosure. Regardless, a composition of the present disclosure may be considered a xenotransplantation product under certain regulatory definitions due to how cells of the composition were historically cultured.

### Cryopreservation

Each of the compositions disclosed herein may be cryopreserved. Methods of suitable cryopreservation are disclosed, for instance, in U.S. Pat. No. 10,091,983, herein incorporated by reference in its entirety. Cryopreserved compositions may comprise glycerin.

After a composition of the present disclosure is thawed following cryopreservation, the composition may secrete a plurality of proteins including human growth factors and cytokines. For instance, a thawed composition may secrete a protein selected from bFGF, GM-CSF, HGF, IL-1α, IL-6, IL-8, IL-10, MMP-1, MMP-3, MMP-9, PIGF, SDF-1α, TGF-β1, and VEGF-A.

### Structural and Functional Properties

The structural and functional properties of the viable bioengineered allogeneic cellularized construct were determined during manufacture and after recovery from cryopreservation. Qualitative assessments of the expression and spatial organization of ECM proteins that would typically be found in intact skin were conducted, and the time course of secretion of cellular factors known to be associated with the inflammation, angiogenesis, proliferation, granulation, and remodeling stages of wound healing were examined.

The structural and functional properties of the construct following cryopreservation were characterized by focusing on extracellular matrix (ECM) molecule expression and secreted protein factors typically associated with acute wound healing in vivo. Allogeneic cellularized skin constructs (i.e. StrataGraft) were prepared, cryopreserved, thawed, and cultured in vitro. ECM protein expression was determined using indirect immunofluorescence on cross sections of the constructs using commercial antibodies against collagen I, III, IV, VI, laminin-332, and decorin. Secretion of soluble protein soluble factors was quantified by multiplex biomarker assays and singleplex ELISA in conditioned media from unmeshed and meshed constructs. Results showed that the cellular components of the viable bioengineered allogeneic cellularized construct deposited ECM proteins with spatial distributions of fibrillar collagens I and III, collagen VI, and decorin indicative of a structurally organized ECM. Spatial distributions of collagen IV and laminin-332 indicated the presence of basement membrane and dermal-epidermal junctions. Soluble protein growth factors, cytokines, and peptides involved in immunomodulation, angiogenesis, and cellular proliferation (bFGF, GM-CSF, HGF, IL-1α, IL-6, IL-8, IL-10, MMP-1, MMP-3, MMP-9, PIGF, SDF-1α, TGF-β1, and VEGF-A) were observed in the pg/cm²/h range at 1 to 168 hours after in vitro reculture. In some examples, the levels of many of these factors may be altered by meshing of the viable bioengineered allogeneic cellularized construct. Thus, the nascent dermal equivalent of the viable bioengineered allogeneic cellularized construct undergoes significant modification, including the production and assembly of major ECM elements of human skin, and the viable cellular components provide sustained secretion of soluble protein factors, including growth factors and cytokines associated with wound healing.

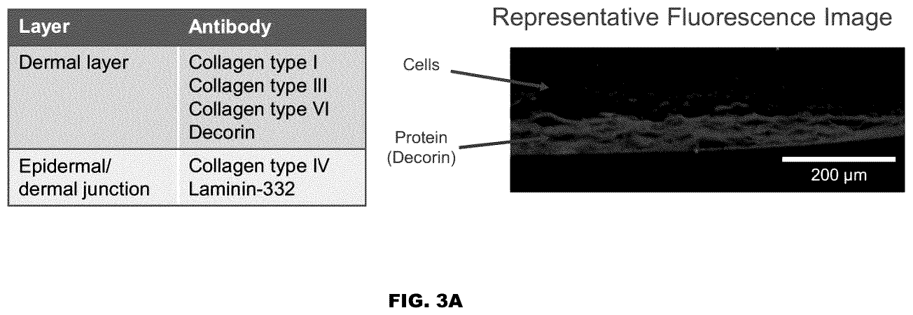

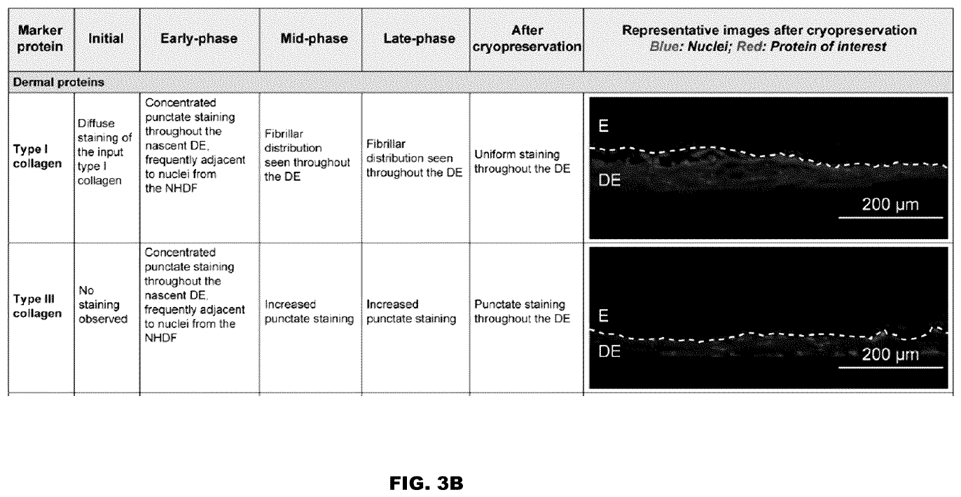

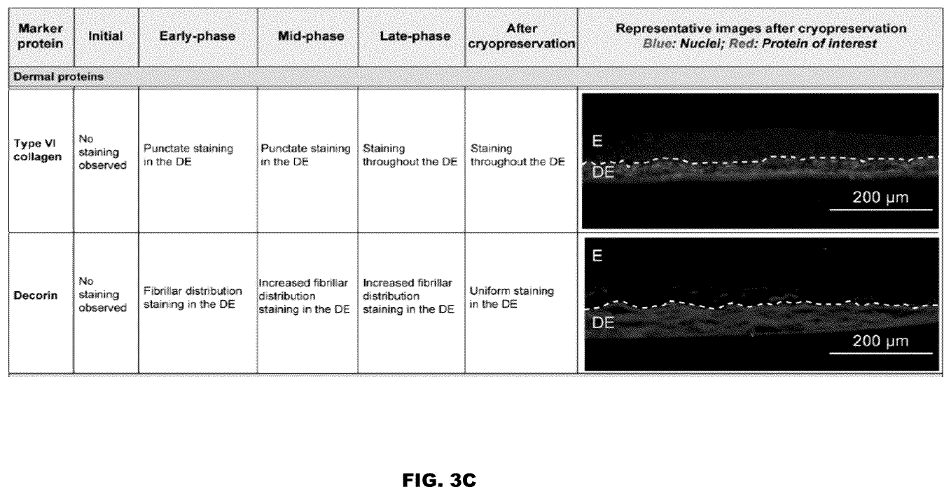

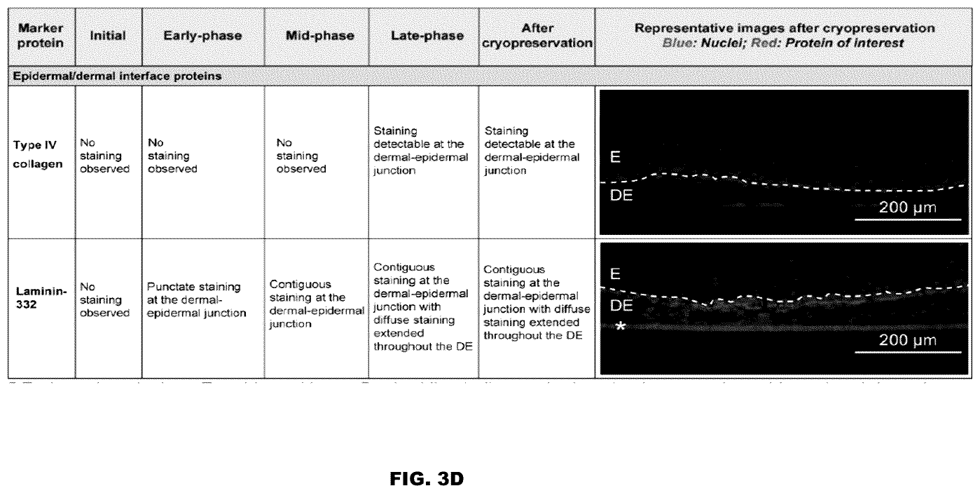

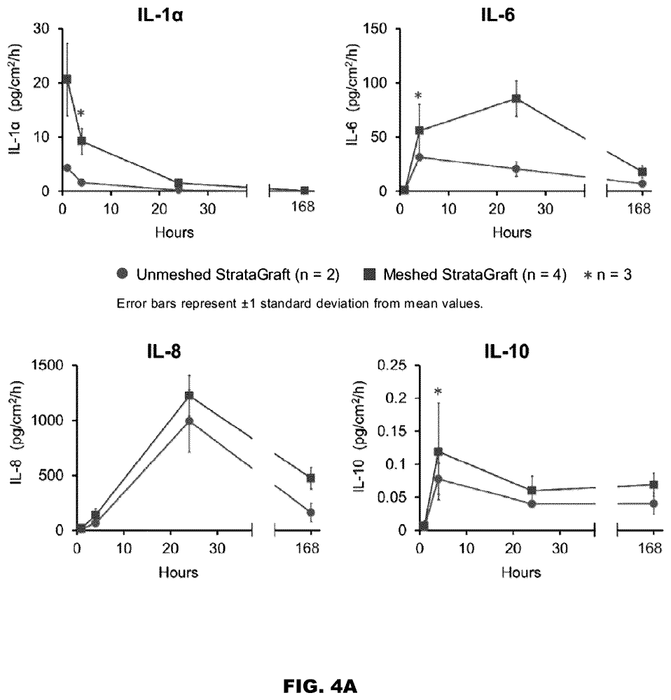

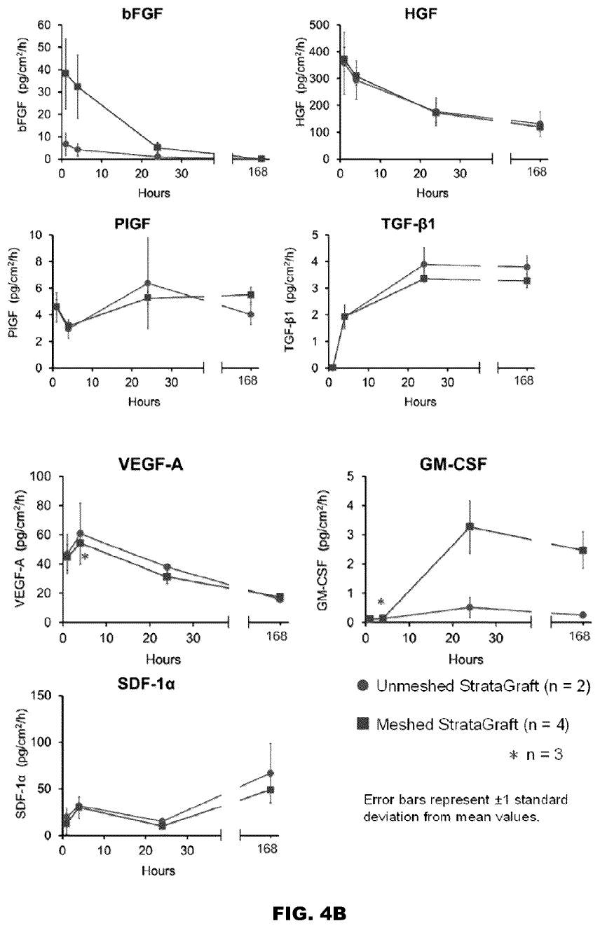

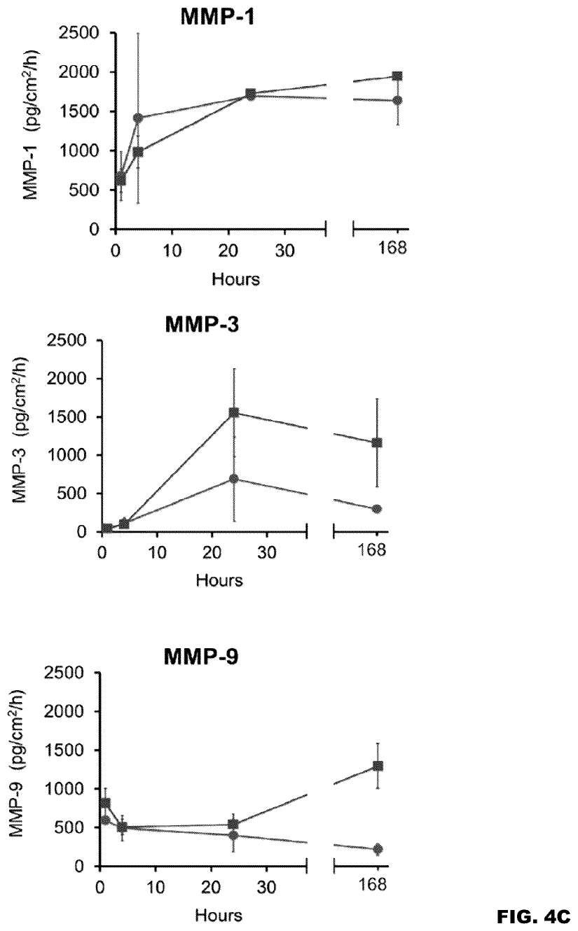

Regenerative wound healing involves a complex series of events that begin with an inflammatory phase that mobilizes immune components and triggers cytokine release. The proliferation stage is characterized by proliferation and migration of cells into the wound environment, which subsequently leads to the synthesis and reorganization of the ECM that is characteristic of the remodeling stage. Serious burn wounds are especially problematic since they often have non-viable eschars that prevents formation of granulation tissue, a vascularized tissue that is rich in fibroblasts. Artificial dermal substitutes provide physical coverings or ECM and cellular constituents that facilitate the proliferation and remodeling phases of wound healing. In various aspects, he viable bioengineered allogeneic cellularized construct has viable keratinocytes and NHDF that synthesize and organize human ECM proteins after thawing and return to culture (FIG. 3) and secrete soluble factors that are associated with regenerative wound healing (FIG. 4).

In humans, reepithelialization is the primary mechanism responsible for the wound closure stage of wound healing. Fibroblasts in the vicinity of the injury differentiate and deposit ECM to help reestablish the three-dimensional layered structure of intact skin. In an embodiment, the allogeneic cellularized construct organizes a stratified ECM reminiscent of intact skin: the presence of type I and type III fibrillar collagens in conjunction with supporting proteins, type VI collagen and decorin indicate a structurally organized dermal layer, and the spatial distribution of type IV collagen and laminin-332 indicate the presence of basement membrane and dermal-epidermal junction (FIG. 3). Thus, upon its application to the wound site, the allogeneic cellularized construct provides an established, organized ECM that accelerates and facilitates reepithelilization, which proves beneficial in injuries such as DPT burn wounds that have large areas of dead cells and damaged ECM tissue.

The viable cellular components of the allogeneic cellularized construct provided immediate and sustained secretion of soluble protein factors, including growth factors and cytokines associated with various stages of wound healing. Following injury, the timeline of secretion of soluble factors by both surviving skin cells as well as invading immune cells involves a complex interplay between these numerous cell types. It is important to bear in mind that as an in vitro system with only NHDF and NIKS cells, the time course and level of soluble factor synthesis and secretion by the allogeneic cellularized construct is not necessarily reflective of the totality of the growth factor secretion levels and temporal patterns in vivo, where much of the secretion is carried out by immune cells and other skin cell types. Nevertheless, the keratinoyctes and fibroblasts in the allogeneic cellularized construct secrete many key factors (Table 2) that induce inflammation, proliferation, granulation, and remodeling of existing matrix that may promote regenerative skin healing at the wound site.

In an embodiment, the allogeneic cellularized construct may secrete soluble factors involved with cell proliferation, inflammation, and formation of granulation tissue such as, but not limited to, GM-CSF, IL-6, IL-8, MMP-3, and MMP-9 (FIG. 4). In the wound healing process, hemostasis occurs immediately after injury, during which a platelet and fibrin clot forms and leads to the secretion of factors such as platelet-derived growth factor (PDGF) and TGF-β. Within 4 days, increased inflammation results in removal of damaged cells, pathogens, and bacteria from the wound area to facilitate tissue repair. The inflammatory phase is characterized by increased secretion of cytokines such as IL-1, IL-6 and growth factors such as bFGF. GM-CSF drives maturation of neutrophils and increases keratinocyte proliferation and neovascularization. IL-6, secreted by keratinocytes and fibroblasts, promotes inflammation and reepithelialization by attracting additional neutrophils and increasing keratinocyte proliferation, respectively. IL-8 promotes the proinflammatory feedback cycle by inducing a neutrophil influx to the wound site. The metalloproteinases (MMP-3 and MMP-9), secreted by keratinocytes, increase cell migration and wound contraction. The next phase of wound healing involves angiogenesis, driven by the release of angiogenic factors such bFGF, PIGF, SDF-1α and VEGF. Cells within the allogeneic cellularized construct may produce several of these key soluble growth factors and cytokines involved in wound healing, suggesting that it may complement or augment the endogenous secretory processes in the wound site.

The concentrations of these secreted factors may be within the typical concentration ranges seen in regenerating wounds. The precise levels and timing of soluble protein factor release in the wound environment may be difficult to measure since it is dependent on a complex interplay of dynamic reciprocity between the ECM and existing cells. However, the viable cellular components of the allogeneic cellularized construct display similar functional attributes to their in situ counterparts.

Secretion of many of these soluble factors may be increased by meshing of the allogeneic cellularized construct (FIG. 4). While meshing in the clinical setting is primarily used to extend the graft size and manage wound exudate, it has been shown to promote keratinocyte migration and proinflammatory cytokine release within 12 to 24 h post-injury. In some embodiments, the increases in secretion of the soluble proteins that promote wound healing may occur in the absence of systemic factors, suggesting that meshing may influence the differentiation or metabolic state of the NHDF and NIKS cells themselves. Observed response in the allogeneic cellularized construct in vitro as a result of meshing represent the anticipated responses from the allogeneic cellularized construct in situ.

The cellular components of the allogeneic cellularized construct deposit and organize human ECM proteins and secrete soluble growth factors and cytokines associated with regenerative wound healing. The organization of the DE and dermal-epidermal junction ECM proteins in a cryopreserved allogeneic cellularized construct is reminiscent of human skin. The viable cellular components of the cryopreserved allogeneic cellularized construct can provide sustained secretion of soluble protein wound healing factors as early as one to four hours after thawing. The manufacture of the provide sustained secretion of soluble protein wound healing factors as early as one to four hours after thawing in a controlled environment, together with its ability to produce and secrete soluble protein factors continuously within a few hours after recovery from cryopreservation, make it an attractive option for promoting regenerative wound healing of severe burns and other complex skin defects.

### Definitions

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, the term “sterile” refers to a skin equivalent that is essentially or completely free of detectable microbial or fungal contamination.

As used herein, the term “NIKS cells” refers to cells having the characteristics of the cells deposited as cell line ATCC CRL-12191. “NIKS” stands for near-diploid immortalized keratinocytes and is a registered trademark.

As used herein, the term “viable” when used in reference to a skin equivalent refers to the viability of cells in the skin equivalent following cryopreservation. In preferred embodiments, a “viable” skin has an A550 of at least 50%, 60%, 70%, 80% or 90% of a control non-cryopreserved tissue as measured by an MTT assay or at least 50%, 60%, 70%, 80% or 90% of the readout value of a similar viability assay.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1: Tissue Manufacture and Organotypic Culture

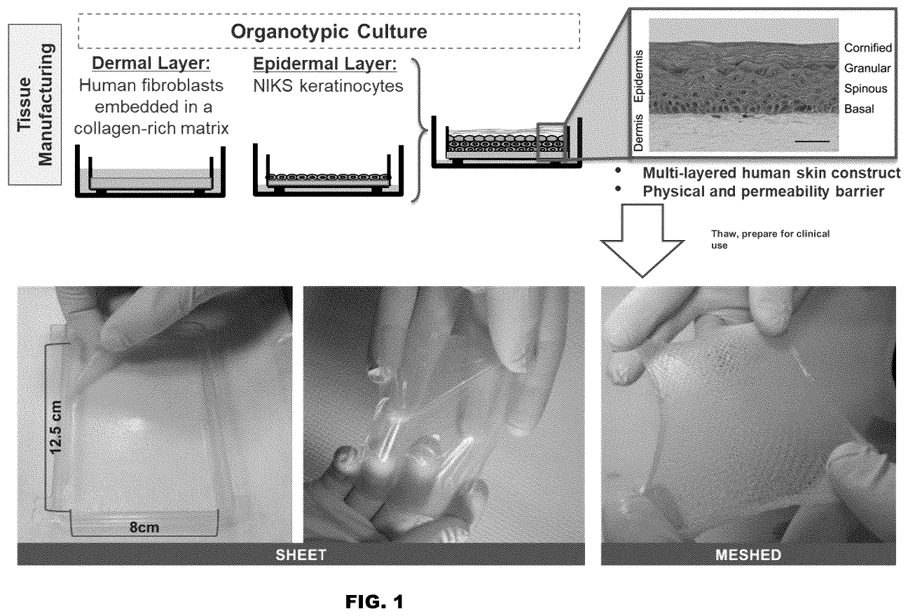

StrataGraft tissue was manufactured as illustrated in FIG. 1. Cryopreserved NHDF and NIKS were thawed and expanded in monolayer culture. NHDF cells were harvested, combined with type I collagen and dispensed into culture vessels to form the dermal equivalent (DE) layer. NIKS keratinocytes were then harvested, plated on the DE, and allowed to develop a fully stratified epidermal layer in organotypic culture. At the end of the manufacturing process, the tissue was treated with cryoprotectant and cryopreserved at −70 to −90° C. until use.

### Example 2: Immunohistochemistry

Indirect immunofluorescence was used to detect ECM proteins on cross sections of StrataGraft using standard techniques. Briefly, developing tissues collected during the production process, as well as the final product tissues and recovered from cryopreservation were fixed for immunostaining. Tissue samples were harvested as 8 mm diameter circular biopsies, fixed in 1% paraformaldehyde in 1× phosphate buffered saline (PBS) for 3 h at 4° C., washed in 1×PBS, then transferred into 20% sucrose. Samples were embedded and frozen in Optimal Cutting Temperature media and stored at −80° C. until processed for cryosectioning at 5 μm thickness. Cryosections were fixed in cold acetone for 5 minutes and air dried. Slides were rinsed with 1×PBS, and nonspecific antibody binding was blocked with 1×PBS containing 3% serum (goat or donkey, matching the species of antibodies used for secondary detection). Slides were incubated for 60 minutes at 37° C. in a humid chamber with primary antibodies in blocking solution at a dilution specific to each antibody (Table 1). Slides were washed twice with 1×PBS and incubated with fluorescent dye-conjugated secondary antibodies for 30 minutes at room temperature. Slides were washed twice with 1×PBS, mounted with Prolong Gold with 4′,5-diamino-2-phenylindole (DAPI) counterstain (Gibco-BRL) and stored overnight at 4° C. in the dark prior to capture of digital images.

TABLE 1
Antibodies

| | | | | |
|---|---|---|---|---|
| Antibody | Immunogen | Species | Vendor | Catalog #/ RRID#^{a} [symbol] |

Proteins associated with the dermal layer

| | | | | |
|---|---|---|---|---|
| Collagen type I, | Full length native protein | rabbit | Abcam, | ab34710/ AB_73168 [symbol] |
| diluted 1:400 | (purified) corresponding to | polyclonal | Cambridge, MA | |
| | Human collagen I aa 1-1464. | | | |
| | Collagen type I from human | | | |
| | and bovine placenta | | | |
| Collagen type III, | Human COL3A1 | goat | Southern | 1330-01/ |
| diluted 1:800 | | monoclonal | Biotech, | AB_2794734 |
| | | | Birmingham, AL | |
| Collagen type VI, | Human type VI collagen | mouse | EMD Millipore, | MAB3303/ |
| diluted 1:400 | | monoclonal | Burlington, MA | AB_11211203 |
| Decorin, diluted | Recombinant fragment | rabbit | Abcam, | ab175404/ NA |
| 1:400 | corresponding to human | polyclonal | Cambridge, | |
| | decorin aa 31-359. | | MA; | |

Proteins associated with the epidermal/dermal junction

| | | | | |
|---|---|---|---|---|
| Collagen type IV, | Full length native protein | mouse | Abcam, | ab6311/ AB_305414 [symbol] |
| diluted 1:100 | (purified) corresponding to | monoclonal | Cambridge, | |
| | Human Collagen IV. | | MA; | |
| Laminin-5 (laminin- | Amino acid residues 382-608 | mouse | EMD Millipore, | MAB19562/ |
| 332) γ2 chain; | of human laminin-5 γ2 chain | monoclonal | Burlington, MA | AB_94454 |
| diluted to 1:100 | (III domain) expressed as a | | | |
| | GST fusion protein | | | |

Control staining for nascent dermal equivalent

| | | | | |
|---|---|---|---|---|
| Collagen type I, | Bovine skin collagen type I | mouse | ThermoFisher, | MA1-26771/ |
| diluted to 1:500 | | monoclonal | Waltham, MA; | AB_2081889 |

^{a}Universal antibody resource number, https://antibodyregistry.org [symbol]
indicates data missing or illegible when filed

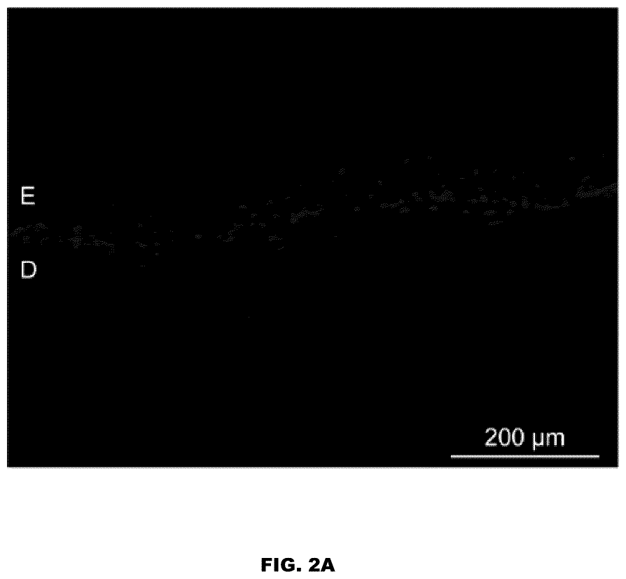

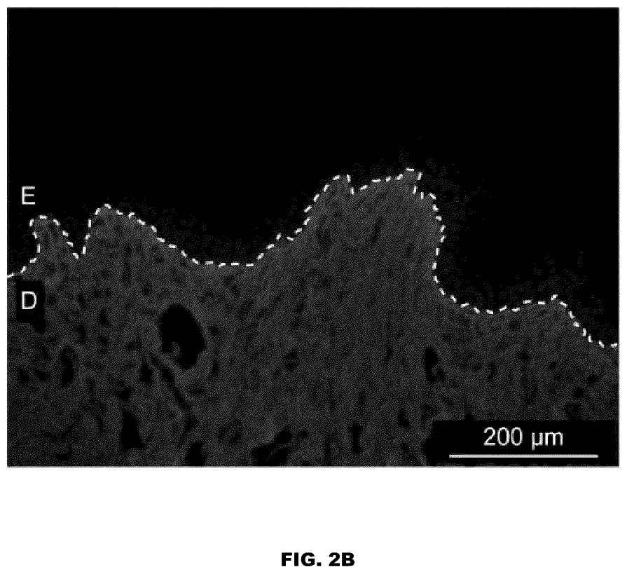

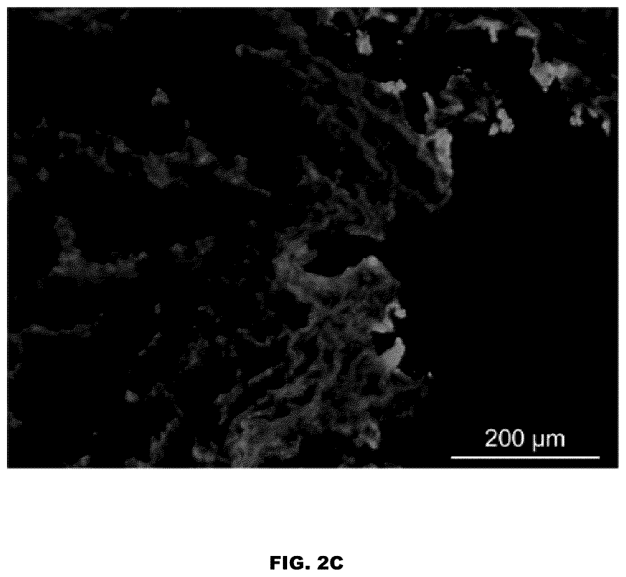

Antibodies against collagens I, III, IV, and VI, and laminin and decorin were obtained from commercially available sources listed in Table 1. Antibody staining was visualized with Alexa 594-conjugated secondary antibodies against the appropriate species; cell nuclei were stained with DAPI. Positive controls for antibody staining were verified with human neonatal foreskin; for negative controls, primary antibodies were omitted. In nascent DE, the presence of input collagen was verified with anti-collagen I (Table 1) visualized with a secondary antibody conjugated to Alexa 488 (FIG. 2). Samples were viewed in cross section under an Olympus IX-71 epifluorescence microscope equipped with CellSens digital imaging software, and images acquired with an Optronics DI 750 CE camera (Coleta, GA) at 200× magnification.

### Example 3: Multiplex Panels and ELISAs

An array of soluble protein factors, typically secreted by fibroblasts and keratinocytes, and associated with different stages of wound healing is provided in Table 2. Protein levels were quantified by multiplex biomarker assays and singleplex enzyme-linked immunosorbent assays (ELISA) in conditioned media collected from unmeshed and meshed StrataGraft. Three cryopreserved tissues from each of two independent lots of StrataGraft were thawed and processed under conditions that approximate clinical handling of tissues prior to patient application. Two tissues from each lot were meshed 1:1 with a Brennan mesher. Meshed and unmeshed tissues were then cultured in fresh media to allow for release of soluble factors into the culture medium. Conditioned media were collected after 1, 4, 24, and 168 hours, and assayed using either Human Matrix Metalloproteinase (MMP) 3-Plex Ultra-Sensitive kits, V-Plex Custom Panels (Meso Scale Diagnostics, Rockville, MD), or Singleplex Quantikine® kits (R&D Systems, Minneapolis, MN) as appropriate for the analytes (Table 2) following manufacturers' instructions. Samples tested for interleukin-6 (IL-6) and interleukin-8 (IL-8) were diluted 1:10, samples tested for vascular endothelial growth factor-A (VEGF-A) were diluted 1:2, and all other samples were tested without dilution. Samples from MMP 3-Plex and V-Plex were processed using a MesoScale Discovery Sector S600 instrument, and data capture and analysis were conducted using the Discovery Workbench V. 4.0.12.1 software package (Meso Scale Diagnostics, Rockville, MD). Quantikine® plates were read on a BioTek Synergy 4 Instrument, and analysis was performed using integrated Gen 5 V2.05.5 software package (BioTek, Winooski, VT). All samples were run in duplicate and compared against a media blank. Analytes were quantified as pg analyte/cm² StrataGraft/h.

TABLE 2
Secreted factors chosen for analysis

| | | Involvement in Stages of Acute Wound Healing | | | | |
|---|---|---|---|---|---|---|
| Secreted | Cell Source | | | | Granulation | |
| Factor | in StrataGraft | Inflammation | Angiogenesis | Proliferation | Tissue | Remodeling |

Interleukins

| | | | |
|---|---|---|---|
| IL-1α | Keratinocyte | x | |
| IL-6 | Fibroblast, | x | x |
| | Keratinocyte | | |
| IL-8 | Fibroblast, | x | |
| | Keratinocyte | | |
| IL-10 | Keratinocyte | x | |

Other peptide factors

| | | | | | | |
|---|---|---|---|---|---|---|
| bFGF | Fibroblast | x | x | | x | |
| HGF | Keratinocyte | | | x | | |
| PIGF | Fibroblast, | | x | | | |
| | Keratinocyte | | | | | |
| TGF-β1 | Fibroblast, | x | x | x | x | x |
| | Keratinocyte | | | | | |
| VEGF-A | Fibroblast | x | x | | | |
| GM-CSF | Keratinocyte | | | x | | |
| SDF-1α | Fibroblast | | x | x | | |

Matrix metalloproteinases

| | | | | |
|---|---|---|---|---|
| MMP-1 | Keratinocyte | | x | x |
| MMP-3 | Keratinocyte | | x | x |
| MMP-9 | Fibroblast, | x | x | |
| | Keratinocyte | | | |

^{a}Assays were Singleplex Quantikine ELISA kits (Singleplex), Human MMP 3-Plex Ultrasensitive Kit (3-Plex), or V-Plex Custom Panels (V-Plex).

bFGF, basic fibroblast growth factor; GM-CSF, granulocyte-macrophage colony-stimulating factor; HGF, hepatocyte growth factor; IL, interleukin; MMP, matrix metalloproteinase; PIGF, placental growth factor; SDF-1α, stromal cell-derived factor 1α; TGF-β1, transforming growth factor β1; VEGF-A, vascular endothelial growth factor A

### Example 4: Growth Characteristics of StrataGraft

Histological analysis of tissue sections stained with hematoxylin and eosin revealed the presence of a well-developed epidermal layer of stratified keratinocytes growing on a DE consisting of NHDF embedded in a gelled collagen matrix (FIG. 1). StrataGraft is manufactured in a rectangular format that has a surface area of approximately 100 cm². StrataGraft has handling characteristics similar to a thin split-thickness skin graft and can be easily meshed as typically done for skin grafts.

### Example 5: Characterization of the Extracellular Matrix

To evaluate ECM protein expression, StrataGraft was immunostained for dermal proteins (collagens I, III, VI, and decorin) and epidermal-dermal junction proteins (type IV collagen and laminin-332) typically found in vivo. FIG. 2 shows positive and negative control collagen I staining using neonatal foreskin (FIG. 2A and FIG. 2B) and positive control staining of input collagen the nascent DE using anti-collagen I visualized with Alexa 488 during manufacture (FIG. 2C). Staining of the neonatal foreskin shows the typical dense fibrillar staining of the fully formed DE in situ. Staining of the input collagen shows the established baseline level of collagen and DE organization prior to further modification over time; at this stage, staining for collagen type III, IV, VI, laminin-332, or decorin is not present (data not shown). FIGS. 3A-3D describe the observed immunostaining at different stages of manufacture and shows representative images after cryopreservation. FIG. 3A shows a representative fluorescence image for the detection of ECM proteins. Indirect immunofluorescence with commercial antibodies was used to detect ECM proteins (collagen, laminin-332, and decorin) on cross sections of StrataGraft. Nuclei were stained with DAPI. FIG. 3B shows StrataGraft synthesized fibrillar collagen types I and III, representing the predominant structural components. FIG. 3C shows human collagens were deposited and organized by the cellular components of StrataGraft, including accumulation of type VI collagen and decorin, which function in vivo to guide assembly of collagen structure. FIG. 3D shows the spatial distribution of collagen IV and laminin-332 indicates that StrataGraft organized a basement membrane zone and dermal-epidermal junction.

At the time of initial NHDF plating within the input collagen gel, the StrataGraft did not contain any newly-synthesized human ECM molecules. From Day 8 through Day 33, there was an overall increase of antigen-specific immunofluorescence staining, demonstrating neosynthesis of human ECM during StrataGraft maturation. As such, in one aspect, the construct comprises newly-synthesized human ECM molecules after approximately one week or 8 days of growth. Staining of fibrillar collagens (types I, III and VI) and decorin representing the predominant structural components of dermal ECM appeared in the early stages of manufacture; this staining was mostly localized to the DE of StrataGraft. Type I collagen levels and distribution in the DE was visualized initially as punctate staining localized to the DE; the collagen staining began to appear more intense and fibrous, suggesting its reorganization into fibrillar structures through the late phase of manufacture. Decorin, the proteoglycan associated with type I collagen fibril assembly, was visible in the DE as fibrillar staining during the early phase and continued to increase in intensity through the late phase. The punctate staining for type III collagen at the early phase was primarily localized adjacent to NDHF nuclei, consistent with de novo synthesis and deposition by the NDHF; the staining increased in intensity but retained the punctate distribution through the late phase. Staining for type VI collagen was punctate in the dermal compartment at the early phase, mirroring expression of types I and III collagen, and developed a contiguous staining pattern through the late phase.

For proteins representing predominantly structural components of the epidermal-dermal junction (type IV collagen and laminin), staining was localized to the region between the DE and the overlying keratinocyte layers. Staining for type IV collagen localized at the epidermal-dermal junction became visible only during the late phase of manufacture. Punctate laminin staining was initially visible at the early phase and became more contiguous and densely localized to the epidermal-dermal junction through the late phase, with diffuse staining that extended throughout the DE. The intense signal at the bottom of the figure represents non-specific staining of the plasticware used to grow the tissue.

Following recovery from cryopreservation, staining intensity and distribution patterns visible at the end of maturation for all ECM proteins were retained. DAPI-stained cells were visible throughout the DE and epidermal layers.

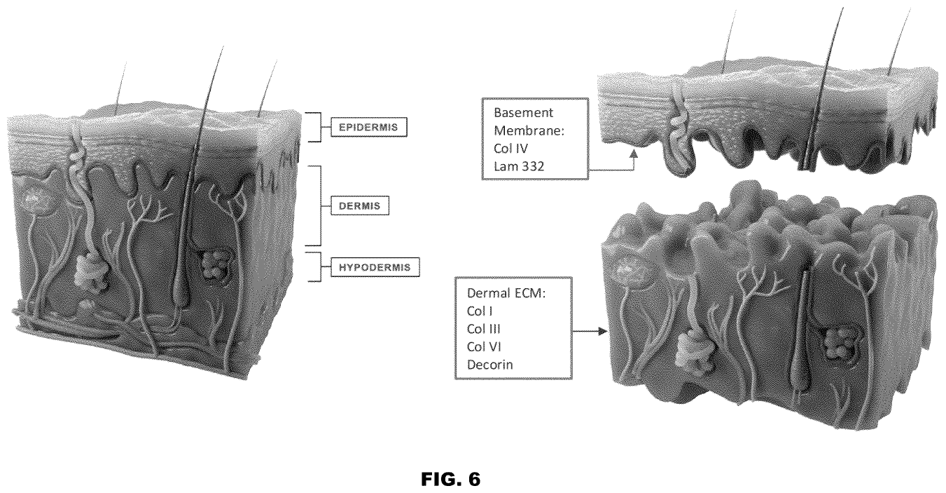

The nascent type I collagen-containing dermal equivalent of StrataGraft undergoes significant modification, including production and assembly of major structural and functional ECM elements of human skin, mediated by the cellular components of StrataGraft. Indirect immunofluorescence demonstrated that the cellular components of StrataGraft deposit human ECM proteins. The presence of type I and type III fibrillar collagens, in conjunction with supporting proteins, type VI collagen, and decorin, indicate a structurally organized ECM. Spatial distribution of type IV collagen and laminin-332 in StrataGraft indicate presence of a basement membrane zone and dermal-epidermal junction. FIG. 6 shows the characterization of the ECM with the components of the basement membrane and dermal ECM.

### Example 6: Wound Healing Factor Secretion

After thawing, the keratinocytes and fibroblasts within StrataGraft produced and secreted growth factors, cytokines, and enzymes involved in immunomodulation, angiogenesis, and cellular proliferation. Interleukins (IL-1α, IL-6, IL-8, IL-10), growth factors (basic fibroblast growth factor [bFGF], hepatocyte growth factor [HGF], placental growth factor [PIGF], transforming growth factor 31 [TGF-β1], and VEGF-A), cytokines (granulocyte-macrophage colony stimulating factor [GM-CSF], stromal cell-derived factor [SDF-1α]) and MMPs (MMP-1, MMP-3, MMP-9) were detected in the pg/cm²/h range in the conditioned media (FIGS. 4A-4C).

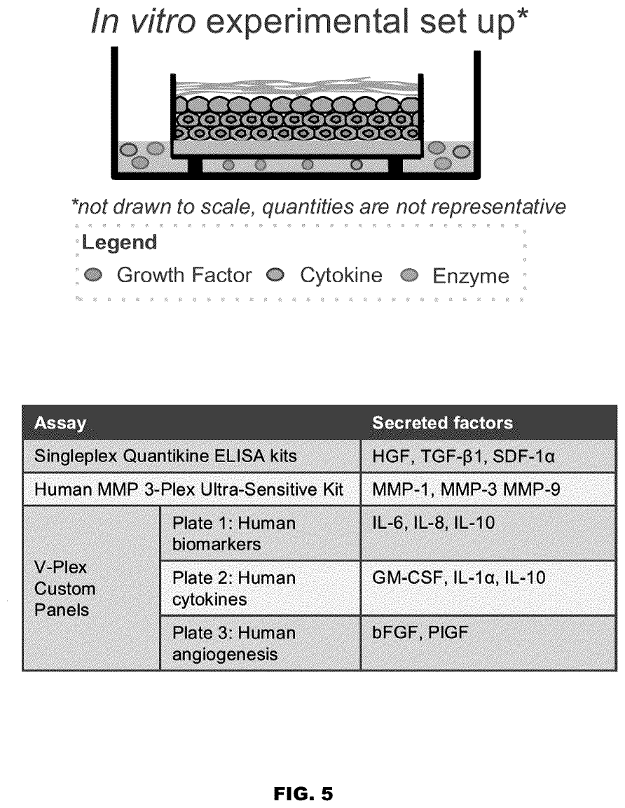

FIG. 5 shows an in vitro experimental setup for measuring biomolecule factor secretion. Protein levels for an array of soluble wound-healing factors were quantified in conditioned media collected from unmeshed and meshed StrataGraft. FIG. 4A shows the results of the secretion of interleukins. After thawing, the cells within StrataGraft continued to produce interleukins at 1 hour, and up to 7 days, after in vitro reculture. Following meshing, expression of IL-1α and IL-6 was greater over the first 24 hours of reculture of meshed compared to unmeshed constructs. FIG. 4B shows the results of the secretion of growth factors and cytokines. After thawing, cells within StrataGraft continued to produce and secrete growth factors and cytokines involved in immunomodulation, angiogenesis, and cellular proliferation. Higher expression of bFGF and GM-CSF were observed in meshed compared with unmeshed constructs. FIG. 4C shows the results of the secretion of matrix metalloproteinases. Changes in the magnitude of MMPs were observed at 1 hour, and up to 7 days, after in vitro reculture. A higher rate of MMP-3 was observed over the first 24 hours of reculture of meshed compared with unmeshed constructs. A more persistent release from meshed construct was observed for MMP-3 and MMP-9.

Results of this analysis revealed dynamic patterns of expression at timepoints between 1 hour and 168 hours after in vitro reculture (FIGS. 4A-4C). In some cases, expression levels were initially low and increased over time in re-culture (e.g., MMP-1 and TGF-β1), while expression of other factors was initially high and decreased overtime (e.g., bFGF, HGF, IL-1α,). Expression of some factors (GM-CSF, MMP-3, IL-6, IL-8) peaked at 24 hours and declined by 168 hours. For some factors, meshed tissues secreted higher levels compared with unmeshed tissue. Higher concentrations of bFGF, IL-1α, IL-6, and MMP-3 were detected over the first 24 hours of reculture in meshed tissues versus with unmeshed tissue, and more persistent release from meshed tissues after extended culture was observed for GM-CSF, IL-6, IL-8, MMP-3, and MMP-9. Secretion of HGF, IL-8, IL-10, MMP-1, PIGF, SDF-1α, TGF-β1, and VEGF-A appeared to be relatively unaffected by meshing.

The viable cellular components of StrataGraft provided sustained secretion of soluble protein factors, including growth factors and cytokines associated with wound healing. Secretion of some factors was increased by meshing.

1. A viable bioengineered allogeneic cellularized construct for topical use at a wound site, the construct comprising allogenic cultured keratinocytes, dermal fibroblasts, and murine collagen, wherein the construct deposits human extracellular matrix (ECM) proteins and secretes soluble factors associated with regenerative wound healing.

2. The construct of claim 1, wherein the construct deposits human extracellular matrix proteins and secretes soluble factors after thawing from cryopreservation.

3. The construct of claim 2, wherein the construct provides sustained secretion of the soluble factors one to four hours after thawing.

4. The construct of claim 1, wherein the secreted soluble factors are human growth factors, cytokines, interleukins, and/or matrix metalloproteinases.

5. The construct of claim 4, wherein the secreted soluble factors induce inflammation, proliferation, granulation, and remodeling of existing matrix to promote regenerative skin healing at the wound site.

6. The construct of claim 1, wherein the secreted soluble factors are selected from the group consisting of bFGF, GM-CSF, HGF, IL-1α, IL-6, IL-8, IL-10, MMP-1, MMP-3, MMP-9, PIGF, SDF-1α, TGF-β1, VEGF-A, and combinations thereof.

7. The construct of claim 5, wherein the secreted soluble factors are present in a range of pg/cm2/h at 1 to 168 hours after in vitro reculture.

8. The construct of claim 1, wherein the construct comprises one or more ECM proteins that are present in intact skin.

9. The construct of claim 1, wherein the construct actively produces human ECM proteins.

10. The construct of claim 1, wherein the construct organizes a stratified ECM substantially similar to intact skin.

11. The construct of claim 18, wherein the human ECM proteins are selected from the group consisting of ECM proteins with spatial distributions of fibrillar collagens I and III, collagen VI, decorin, laminin-332, and combinations thereof.

12. The construct of claim 11, wherein the construct comprises a structurally organized dermal layer.

13. The construct of claim 12, wherein the construct comprises type I and type III fibrillar collagens in conjunction with supporting proteins, type VI collagen, and decorin.

14. The construct of claim 11, wherein the construct comprises a basement membrane and dermal-epidermal junction.

15. The construct of claim 14, wherein the construct comprises a spatial distribution of type IV collagen and laminin-332.

16. The construct of claim 1, wherein the secretion of the soluble factors is increased after meshing the allogenic cellularized construct.

17. The construct of claim 1, wherein the dermal fibroblasts are human dermal fibroblasts.

18. The construct of claim 1, wherein the construct further comprises glycerin.

19. The construct of claim 1, wherein the construct comprises newly-synthesized human ECM molecules after approximately one week or 8 days of growth.

20. A method of treating an adult patient for deep partial-thickness (DPT) burns comprising applying an allogeneic cellularized construct to a DPT wound site, the allogenic cellularized construct comprising allogenic cultured keratinocytes, dermal fibroblasts, and murine collagen, wherein the allogeneic cellularized construct deposits human ECM proteins and secretes soluble factors associated with regenerative wound healing.

21. The method of claim 20, wherein the allogeneic cellularized construct provides an organized ECM that increases reepithelilization.

22. The method of claim 20, further comprising meshing the allogenic cellularized construct prior to applying the allogeneic cellularized construct to the DPT wound site.

23. The method of claim 20, further comprising thawing a cryopreserved allogeneic cellularized construct to applying the allogeneic cellularized construct to the DPT wound site.

24. The method of claim 23, wherein the allogeneic cellularized construct provides sustained secretion of the soluble factors one to four hours after thawing.

25.-27. (canceled)
